(19) United States
(12) Patent Application Publication
(10) Pub. No.: US 2026/0360490 A1
(43) Pub. Date: Nov. 12, 2026

(54) MEDICAL CONSULTATION SYSTEM AND MEDICAL CONSULTATION METHOD

(71) Applicant: MEGAFORCE COMPANY LIMITED, New Taipei City (TW)

(72) Inventors: CHIH-HAN YEN, New Taipei City (TW); Xiu-Lin Chao, New Taipei City (TW); GANG-FENG HO, New Taipei City (TW); YI-WUN TSAI, New Taipei City (TW); BING-HONG CHEN, New Taipei City (TW)

(21) Appl. No.: 19/300,636

(22) Filed: Aug. 14, 2025

(30) Foreign Application Priority Data
May 6, 2025 (TW) 114116879

(51) Int. Cl. G16H 15/00; G16H 10/20; G16H 10/60; G16H 50/20; G16H 50/30; G16H 50/70; G16H 70/60

(52) U.S. Cl. CPC G16H 15/00; G16H 10/20; G16H 10/60; G16H 50/20; G16H 50/30; G16H 50/70; G16H 70/60

(57) ABSTRACT

A medical consultation method includes: configuring a user device to obtain health data of a user; configuring a storage device to store a data warehouse module, a personal data module and a medical assistance model; and configuring a processing device to perform the following processes of: receiving a query text inputted by the user; determining whether the query text includes a medical issue through the medical assistance model; and in response to determining that the query text includes the medical issue, determining, through the medical assistance model and based on a medical-related dataset and the health data, one or more of related diseases, related symptoms and related medical departments that are associated with the medical issue, and generating a response text.

### CROSS-REFERENCE TO RELATED PATENT APPLICATION

This application claims the benefit of priority to Taiwan Patent Application No. 114116879, filed on May 6, 2025. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a system and a method, and more particularly to a medical consultation system and a medical consultation method.

### BACKGROUND OF THE DISCLOSURE

Conventional personal health systems have obvious problems. For example, the conventional personal health systems are only responsible for recording data, and most medical software applications only provide services and suggestions based on information of the general population. Therefore, the conventional personal health systems are not comprehensive systems and cannot effectively help users understand their own physical conditions.

In addition, the conventional personal health systems are unable to continuously learn unique physiological data of each of their users, update and optimize an analysis model, and protect the privacy of their users. Furthermore, the conventional personal health systems are unable to integrate real-time health data of their users with focus points, medical records and daily life records of the user to provide more personalized health advice for the user.

### SUMMARY OF THE DISCLOSURE

In response to the above-referenced technical inadequacies, the present disclosure provides a medical consultation system and a medical consultation method, which are able to perform adjustment according to specific health state of the user and provide privacy protection to the user, thereby addressing the problems in the relevant art.

In order to solve the above-mentioned problems, one of the technical aspects adopted by the present disclosure is to provide the medical consultation system. The medical consultation system includes at least one user device, a storage device and a processing device. The at least one user device is configured to obtain health data of a user. The storage device is configured to store a data warehouse module, a personal data module and a medical assistance model. The data warehouse module is configured to store a medical-related dataset and the personal data module is configured to store the health data. The processing device is connected electrically to the storage device. The processing device is configured to receive a query text inputted by the user and determine whether or not the query text includes a medical issue through the medical assistance model. When the query text includes the medical issue, the processing device, through the medical assistance model and based on the medical-related dataset and the health data, determines one or more of at least one related disease, at least one related symptom and at least one related medical department that are associated with the medical issue, and generates a response text.

In order to solve the above-mentioned problems, another of the technical aspects adopted by the present disclosure is to provide the medical consultation method. The medical consultation method includes processes of: configuring at least one user device to obtain health data of a user; configuring a storage device to store a data warehouse module, a personal data module and a medical assistance model, storing a medical-related dataset in the data warehouse module, and storing the health data in the personal data module; and configuring a processing device connected electrically to the storage device to: receive a query text inputted by the user, determine whether or not the query text includes a medical issue through the medical assistance model, and in response to determining that the query text includes the medical issue, through the medical assistance model and based on the medical-related dataset and the health data, determining one or more of at least one related disease, at least one related symptom and at least one related medical department that are associated with the medical issue, and generating a response text.

These and other aspects of the present disclosure will become apparent from the following description of the embodiment taken in conjunction with the following drawings and their captions, although variations and modifications therein may be effected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a block diagram of a medical consultation system according to one embodiment of the present disclosure;

FIG. 2 is a flowchart diagram of a medical consultation method according to one embodiment of the present disclosure;

FIG. 3 is a schematic diagram of a process of establishing a medical-related dataset according to one embodiment of the present disclosure;

FIG. 4 is a detailed flowchart diagram of process S13; and

FIG. 5 is a flowchart diagram of a medical consultation method according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

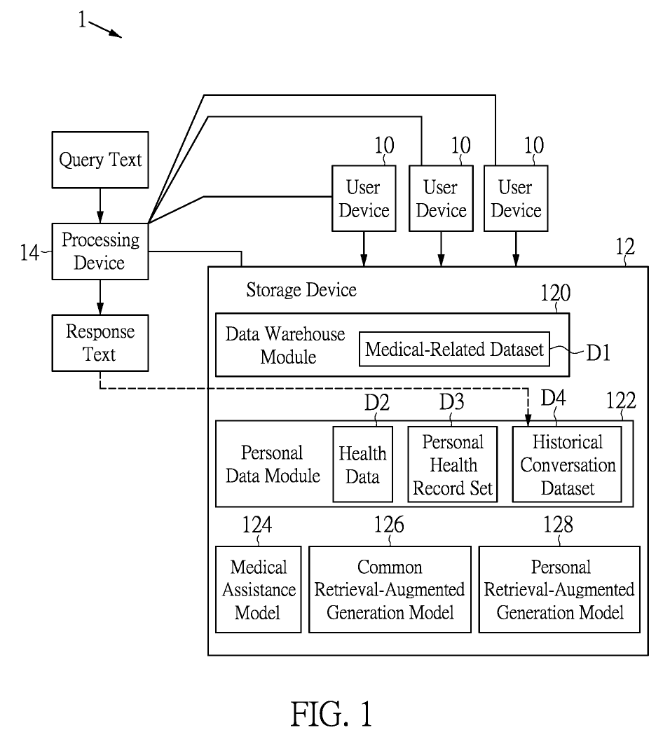

Reference is made to FIG. 1, which is a block diagram of a medical consultation system according to an embodiment of the present disclosure. A medical consultation system 1 includes a plurality of user devices 10, a storage device 12 and a processing device 14.

Each of the plurality of user devices 10 may be configured to obtain health data of a user. Each of the plurality of user devices 10 may be a terminal apparatus having communication and display functions. For example, each of the plurality of user devices 10 may be a smart phone, a tablet computer, a notebook computer, or a wearable device (such as a smart watch, a bracelet, a ring necklace, a headset, etc.). Each of the plurality of user devices 10 may continually receive and record a plurality of pieces of biometric data of the user. The plurality of pieces of biometric data received by each of the plurality of user devices 10 may include a heart rate, a heart rate variability, a respiratory rate, a blood oxygen saturation, a blood pressure, a body temperature, galvanic skin response, number of steps, calorie consumption, an amount of activity, posture and movement, fall events, sedentary events, total sleep time, sleep stages (deep sleep, light sleep, and REM sleep), sleep onset and wakefulness time, a sleep quality score, body surface humidity or sweat response, wrist tilt and other posture angles, GPS positioning information, an ambient noise intensity, blood sugar variation (that is measured by medical sensors), and so on. These biometric data can be provided as diverse health monitoring information in real time, which is conducive to subsequent personalized health analysis and risk assessment.

Furthermore, in some embodiments, the user device 10 may have communication and display functions, and provide a text input interface configured to receive user query information. In addition, the user device 10 may also display a medical consultation response provided by the system, so that the user is able to perform interactive health management.

The storage device 12 may be a server-type storage unit, a network attached storage device (NAS) or a storage server built into a cloud platform, and has high capacity and high reliability characteristics. The storage device 12 may store various data modules such as a data warehouse module 120, a personal data module 122 and a medical assistance model 124 that are configured to support retrieval and generation of subsequent consultation contents.

The data warehouse module 120 is configured to store a medical-related dataset D1. The personal data module 122 is configured to store health data D2 and a personal health record set D3. For example, the health data D2 may include the health data obtained by the user device 10. The processing device 14 collects health checkup information and medical history data of the user and accordingly establishes the personal health record set D3.

In addition, the health data D2 further includes a plurality of personalized normal ranges that are established based on the plurality of pieces of biometric data by the processing device 14. The personalized normal range is a data range that is established by observing and gathering statistics on physiological characteristics and daily conditions of specific users over a long period of time. The personalized normal range is different from a general population average value. The system may perform time series analysis and distribution statistics on each one of the plurality of pieces of biometric data (such as a heart rate, blood pressure, blood oxygen saturation, a body temperature, heart rate variability, sleep hours, and so on) within its reasonable variation range for constructing a normal value range that conforms to an individual daily variation pattern.

For example, over the past three months, a resting heart rate of the user can be almost between 58 bpm and 62 bpm in the morning, and, on average, rise to 85 bpm after the user exercises. The system, based on these daily physiological change trends of the user, establishes two personalized normal heart rate ranges: 58-62 bpm of the user resting and 80-90 bpm of the user that has exercised. Similarly, if the body temperature of the user is slightly increased in a particular season or the heart rate variability of the user is reduced under pressure, the personalized normal range of the user is automatically changed with differences in the body temperature and the heart rate variability of the user through statistical modeling.

These establishment of the personalized normal ranges is not only useful for real-time abnormality detection, but also useful for preventing misjudgments caused by general population standards, thereby improving accuracy and reliability of personalized health monitoring performed by the system. Such personalized normal ranges are dynamically updated and adjusted based on collected new data for further reflecting physical changes and health trends of the user.

Furthermore, the personal data module 122 collects a plurality of historical query texts that are inputted by the user and a plurality of historical response texts generated through the medical assistance model 124 to generate and store the historical conversation dataset D4.

In some embodiments, the medical consultation system 1 includes the plurality of storage devices 12, and the data warehouse module 120 and the personal data module 122 are stored respectively in different ones of the plurality of storage devices 12. For example, in order to ensure privacy, all collected personal information, including health records and chat topics of the user, is only stored in the storage device 12 at a local terminal and is only allowed to be accessed by an owner of the storage device 12.

The processing device 14 may be electrically connected to the storage device 12 and the plurality of user devices 10. For example, the processing device 14 may be communicated with the storage device 12 and the plurality of user devices 10 in a wired or wireless manner, and may access various data modules in the storage device 12 and/or receive input data and the health data from the user device 10. The processing device 14 may be a central processing unit (CPU) server, a graphics processing unit (GPU) server, or an edge computing device including an acceleration module, and is configured to execute system computing and artificial intelligence inference processes.

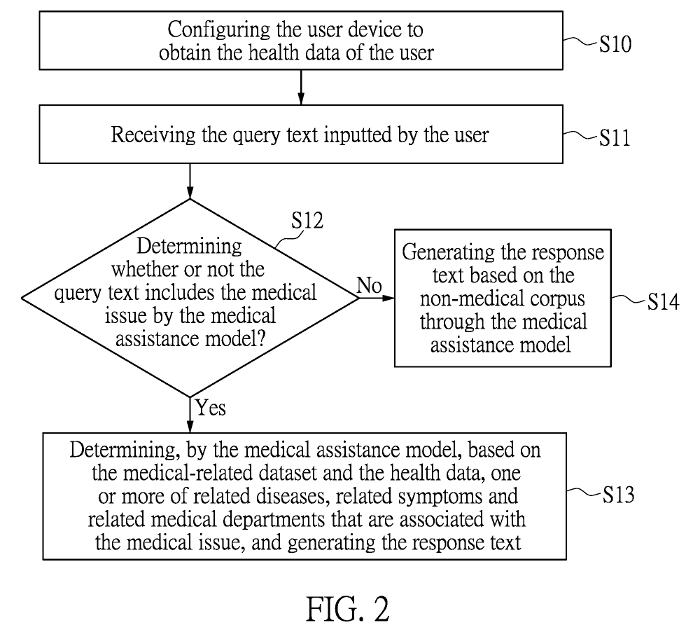

In one embodiment, the present disclosure further provides a medical consultation method, which is applicable to the medical consultation system 1 described above. FIG. 2 is a flowchart diagram of a medical consultation method according to an embodiment of the present disclosure.
As shown in FIG. 2, the medical consultation method may include: process S10 of configuring the user device to obtain the health data of the user;
process S11 of configuring the processing device 14 to receive the query text inputted by the user;
process S12 of determining whether or not the query text includes the medical issue through a medical assistance model by the processing device 14; and
in response to determining that the query text includes the medical issue through the medical assistance model by the processing device 14 in process S12,
in process S13, determining, based on the medical-related dataset and the health data, by the processing device 14, one or more of related diseases, related symptoms, related medical departments that are associated with the medical issue, and generating a response text.

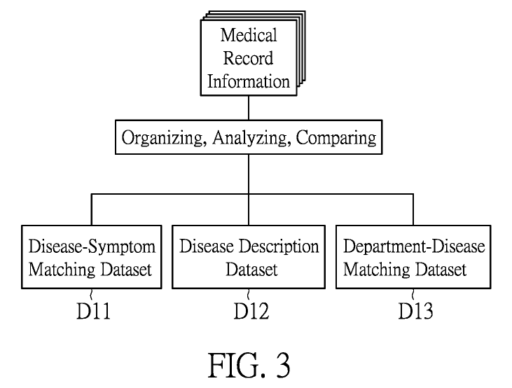

The following lines describe how the medical-related dataset D1 is established. Reference is made to FIG. 3, which is a schematic diagram of a process of establishing a medical-related dataset according to an embodiment of the present disclosure. In an establishment process of medical dataset D1, a plurality of pieces of actual or de-identified medical record information (including texts and voice) may be collected. The medical record information may obtained from electronic medical record systems of medical institutions, open medical databases, medical journals, clinical guidelines, health insurance claims records, and so on. By systematically organizing and structuring those data, field information such as disease names, related symptom descriptions, clinical diagnosis results, treatment manners, medical codes and the medical departments may be obtained.

Further, the field information is analyzed and compared with medical ontology for establishing various structurally related sub-datasets through natural language processing techniques (such as entity recognition and semantic association analysis techniques). The structurally related sub-datasets include: a disease-symptom matching dataset D11, which is configured to record association between specific diseases and common clinical symptoms thereof; a disease description dataset D12, which is configured to organize information such as definition, cause, pathogenesis and risk factors of each of the diseases; and a department-disease matching dataset D13, which is configured to classify each of the diseases into its corresponding diagnosis and treatment departments (for example, respiratory diseases belong to a thoracic medicine department, heart diseases belong to a cardiology department, and so on).

The various structurally related sub-datasets established through the above processes are constituted together to form the medical-related dataset D1, which can be used for subsequent training, inference and knowledge retrieval sources of consultation response contents of the medical assistance model 124. The medical-related dataset D1 may be updated regularly as needed for reflecting latest clinical research results and medical practice information.

Therefore, in process S11, when the processing device 14 receives the query text inputted by the user, the processing device 14 may be configured to activate the medical assistance model 124. The medical assistance model 124 is, for example, a natural language processing (NLP) model, which may be configured to analyze semantic features of the inputted query text, including word segmentation, part-of-speech tagging, named entity recognition, and semantic embedding. By performing these pre-processing processes, the medical assistance model 124 is configured to identify words that have medical connotations such as body parts, symptom names, disease names, examination behaviors and drug names in the inputted query text.

Then, the semantic features of the inputted query text are compared with the medical-related dataset D1 through the medical assistance model 124. Specifically, the system may compare the words or semantic vectors in the inputted query text with medical entities in the disease-symptom matching dataset D11 and the disease description dataset D12 to calculate similarity. If the similarity reaches a preset threshold, the system may determine that the inputted content involves the medical issue. For example, if the content inputted by the user includes “I've been coughing a lot and I have a sore throat recently”, “coughing” and “sore throat” are compared with corresponding respiratory symptom data in the medical-related dataset D1, and accordingly, this query text is determined as a medically relevant inquiry.

In some embodiments, the storage device 12 further stores a common retrieval-augmented generation model 126 and a personal retrieval-augmented generation model 128 for assisting the medical assistance model 124 in generating the response text. The common retrieval-augmented generation model 126 and the personal retrieval-augmented generation model 128 are retrieval-augmented generation (RAG) type models, a core architecture of which usually includes two portions that are a retriever and a generator. The common retrieval-augmented generation model 126 and the personal retrieval-augmented generation model 128 may have different model architectures according to application requirements.

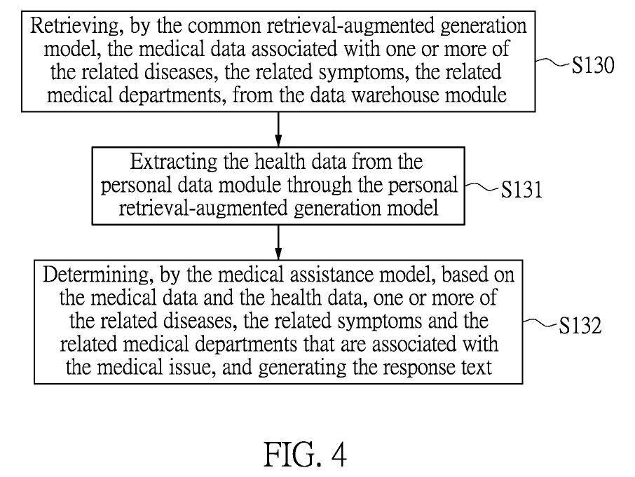

Reference is made to FIG. 4, which is a detailed flowchart diagram of process S13. The process S13 may further include processes 130 to 132 that are performed by the processing device 14.

In process S130, the processing device 14, through the common retrieval-augmented generation model 126, retrieves medical data associated with one or more of the related diseases, the related symptoms, the related medical departments from the data warehouse module 120.

In process S130, the processing device 14, through the common retrieval-augmented generation model 126, converts the query text into a first semantic vector, and performs similarity comparison on the first semantic vector set and the medical-related data set D1 of the data warehouse module 120 for retrieving a plurality of first data segments related to the medical issue and providing the plurality of first data segments to the medical assistance model 124.

In detail, the processing device 14, through the common retrieval-augmented generation model 126, based on key semantic information of the inputted query text (such as the symptom descriptions inputted by the user, possible disease or related department analyzed by the system), codes, embeds and converts the semantics of the query text or the symptom information into a semantic vector, performs vector-based retrieval on the semantic vector in a vector format, and performs similarity comparison on the semantic vector and the medical-related dataset D1 pre-established in the data warehouse model 120 for searching a medical document or a knowledge fragment that is closest to the semantic of the inputted query text. For example, the searched medical document or the knowledge fragment may include a matching relationship between the disease and the symptom, the disease descriptions and the pathogenesis, common diagnostic procedures and treatment recommendations, and classification rules of the diseases and the medical departments corresponding thereto, but the present disclosure is not limited thereto. Then, the processing device 14, through the common retrieval-augmented generation model 126, performs retrieval and preprocessing of contents of the medical document or the knowledge fragment, obtains paragraphs where the contents are stated, and performs preliminary filtering and sorting of the contents according to context of the medical document or the knowledge fragment for generating and providing subsequent contents to the medical assistance model 124.

In process S131, the processing device 14 obtains the health data from the personal data module 122 through the personal retrieval-augmented generation model 128.

In process S131, the processing device 14 may, through the personal retrieval-augmented generation model 128, continually use the first semantic vector set that is generated by converting the query text in process S130, or convert the query text into a second semantic vector set (based on a synchronous processing architecture) and only use the second semantic vector set. In addition, the processing device 14 performs similarity comparison on the second semantic vector set and the personal data module 122 for retrieving a plurality of second data segments related to the medical issue from the personal data module 122, and provides the plurality of second data segments to the medical assistance model 124.

In more detail, the processing device 14 may encode historical health data (such as records of changes in a heart rate, a blood pressure, a body temperature, etc.), conversation records (past symptom descriptions, consultation records and medical advice) and personal preferences and response habits (such as types of questions that are often asked and suggestions that have been rejected, etc.) into vectors, and may store these vectors in the personal data module 122. The processing device 14 may, through the personal retrieval-augmented generation model 128, obtain data segments most relevant to the query text based on the similarity comparison of the semantic. The personal information obtained by the personal retrieval-augmented generation model 128 and the information generated by the common retrieval-augmented generation model 126 are provided to the medical assistance model 124 as a conditional context for generating the response text including personalized medical advice.

In process S132, the processing device 14, through the medical assistance model, based on the medical data and the health data, determines one or more of the related diseases, the related symptoms and the related medical departments that are associated with the medical issue, and generates the response text.

In process S132, the information retrieved by the common retrieval-augmented generation model 126 and the personal retrieval-augmented generation model 128 are used as condition input information of the medical assistance model 124, and the context is constructed based on the condition input information and the query text of the user through the medical assistance model 124. Specifically, the medical assistance model 124 assigns weights based on semantic relevance, importance and timeliness of a retrieved result, prioritizes to use personalized information (such as diseases known by the user and past symptom patterns) to perform fine-tune on the response content, and uses common medical knowledge as supplementary or corroboration information. On this basis, for example, in the medical assistance model 124, a generative language model (such as a fine-tuned GPT or T5 architecture) may be used for natural language generation (NLG) for organizing the response text in a medically professional and easy-to-understand manner. The response text may include a preliminary explanation of the symptoms, key points that the user is advised to observe, the departments that may be involved, and whether immediate medical attention is recommended. Finally, the system sends the generated response text to the user device for providing the instant and personalized medical advice.

More specifically, the medical assistance model 124 is configured to, through the personal retrieval-augmented generation model 128, based on the information such as the health data D2, personal health record set D3, the historical conversation dataset D4 and the personalized normal ranges that are stored in the personal data module 122, adaptively adjust the determination criteria used for generating the response text. In addition, through the medical assistance model 124, the common retrieval-augmented generation model 126 and the personal retrieval-augmented generation model 128, a personalized interactive experience are provided without retraining the model for each of the users.

As shown in FIG. 2, in response to determining that the query text does not include the medical issue in process S12, process S14 is performed. In process S14, the response text is generated based on non-medical corpus through the medical assistance model 124.

For example, when the query text is determined to not include the medical issue by the medical assistance model 124, a non-medical response process is performed for ensuring that the user receives an appropriate and interactive response instead of a system error response or no response.

For example, if the inputted content includes daily greetings (such as “Hello”), system operation inquiries (such as “How do I use this system? ”), emotional statements (such as “I am very tired today”), or other contents that do not have clear health appeals, the inputted content is determined to be classified into a non-medical category through the medical assistance model 124. Under this condition, disease inference and medical information retrieval processes are not performed, but a pre-designed non-medical corpus or a coping strategy module is used for generating appropriate corresponding texts.

For example, if “How can you help me?” or “I'm a little tired but not uncomfortable” is inputted by the user, the response text that may be used for maintaining interaction with the user and naturally guiding the user back to a health topic may be generated by a model, such as “I can help analyze your health data and provide advice” or “If you’re feeling tired, take a look at your recent sleep or heart rate patterns” and the like.

In addition, if the inputted content is related to operation of the system, function descriptions, usage instructions or recommended processes may be returned by the model, such as “You can click icon below to start uploading health data” or “If you want to query past conversation records, please click the history button in upper right corner”. Through this classification process and contextual response generation, a natural conversation flow and positive interaction experience, even in situations where there are no medical issue, can be maintained by the medical assistance model 124.

It should be noted that, training of the medical assistance model 124 may be divided into medical semantic understanding and question classification training and response generation ability training. First, in order to make the medical assistance model 124 accurately determine whether the content inputted by the user involves the medical issue, supervised learning must be performed by using an annotated corpus. The annotated corpus must include a plurality of pieces of real or simulated query sentences, each is marked with a annotation representing whether it is related to medical and the category such as the symptom, the disease or the department that is involved by it. The medical assistance model 124 may be fine-tuned based on one or more pre-trained language models such that the medical assistance model 124 is able to recognize medical sentences and understand semantics. In addition, the annotated corpus may further include named entity recognition (NER) and symptom entity annotation for improving ability of analyzing the symptoms and medical terms.

In terms of response generation, the medical assistance model 124 must be trained by combining retrieval and generative architectures. By using the medical-related dataset D1 as a knowledge source, combined with the medical knowledge provided by the common retrieval-augmented generation model 126 and the personalized information provided by the personal retrieval-augmented generation model 128, training samples corresponding to input conditions and reference texts are constructed. Then, the medical assistance model 124, through a generative model such as a GPT architecture, is trained to generate semantically consistent and medically professional answers based on the context. In order to improve accuracy and practicality of the medical assistance model 124, standard answers reviewed by doctors may also be imported as a basis for fine-tuning, and interaction records of the users may also be continuously collected in later stages for reinforcement learning and feedback-based adjustments.

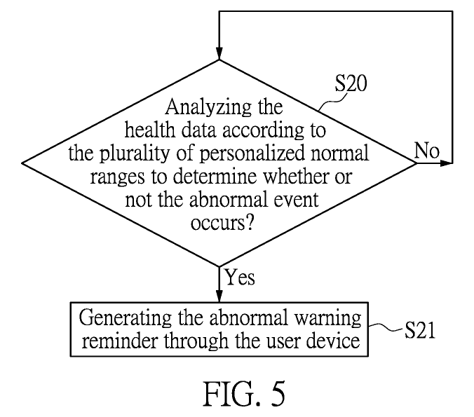

Reference is made to FIG. 5, which is a flowchart diagram of a medical consultation method according to an embodiment of the present disclosure. As shown in FIG. 5, the medical consultation method further includes: configuring the processing device 14 to perform processes S20 and S21.

In process S20, the health data is analyzed based on the plurality of personalized normal ranges for determining whether the abnormal event occurs. Specifically, the processing device 14 may compare each piece of newly received data (such as a current heart rate, a current body temperature, current blood pressure or a current sleep index) with the corresponding personalized normal range of the user in real time. If the newly received biometric data of the user exceeds a variation allowable range defined based on the personalized normal range of the user (for example, the heart rate is more than 10% of a normal upper limit value of the resting heart rate of the user, or the blood pressure suddenly drops below a daily blood pressure average value of the user), the system determines that the newly received biometric data may represent a potential abnormal event.

In some embodiments, in order to improve accuracy, the processing device 14 determines the abnormal event, based on auxiliary data such as changing trend over a continuous period of time, linkage relationships between different physiological parameters (for example, a linkage relationship between the blood pressure and the heart rate being increased with a reduction in the blood pressure), and consistency of previous and subsequent data. By this mechanism including personalized thresholds and multi-parameter analysis, sensitivity and accuracy of abnormality identification are effectively improved.

If the abnormal event is determined to occur in process S20, an abnormal warning reminder is generated by the user device in process S21. For example, when the processing device 14 determines that the current physiological state of the user deviates from the personalized normal range thereof and identifies it as the abnormal event based on preset determination conditions (such as abnormal duration, variation range or cross-indicator confirmation), a warning mechanism is triggered. A warning message may be sent though a real-time push notification, a sound reminder, a vibration reminder, or a screen pop-up window for reminding the user to pay attention to his or her own condition by the user device 10 (such as a smart phone or a smart watch). The warning message may include contents such as an abnormal item name (such as “a too-high heart rate”), detection time, an abnormal data value, comparison information including a normal range, and even include a brief action suggestion (such as “recommend to rest”, “if discomfort persists, please contact medical personnel”, etc.). In addition, the processing device 14 may also automatically synchronize abnormal event records to a personal health record module according to settings, and incorporate them into references when the processing device 14 interacts with the medical assistance model later. In addition, a determination manner of the abnormal event may be divided into a short-term determination manner and a long-term determination manner for analysis and warning. In the short-term determination manner, if data for the day is different from results collected within a predetermined time period, the abnormal warning reminder should be issued. If the data tracked over a long period of time gradually deteriorates, for example, an average blood pressure in a current month is higher than an average blood pressure three months ago, the abnormal warning reminder should also be issued.

If the abnormal event is determined to not occur in process S20, process S20 is repeated.

### Beneficial Effects of the Embodiments

One of beneficial effects of the present disclosure is that, the present disclosure provides the medical consultation system and the medical consultation method that are able to continuously receive and record the biometric data through integration with the wearable device. The personal health data and the data records are able to be continuously collected for thelearning of the medical assistance model, and the medical assistance model is able to be adjusted based on specific health conditions of the user for providing more appropriate responses. Furthermore, the personalized normal value ranges are able to be established based on the biometric data for detection of the abnormal events.

In addition, during interaction between the user and the medical assistance model, issues and concerns of the users are learned by the medical assistance model, and a correlation between this information and the recorded health values is analyzed for actively assisting the user in health monitoring and recommending medical treatment.

Furthermore, the personal data module is able to be established and continuously updated through real-time data from the wearable device, input of the user, the health checkup information and the medical history. When the user interacts with the medical assistance model, cross analysis is performed on the historical medical records and the current health states of the user from the personal data module by the medical assistance model.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A medical consultation system, comprising:
at least one user device configured to obtain health data of a user;
a storage device configured to store a data warehouse module, a personal data module and a medical assistance model, wherein the data warehouse module is configured to store a medical-related dataset and the personal data module is configured to store the health data; and
a processing device electrically connected to the storage device, wherein the processing device is configured to receive a query text inputted by the user and determine whether or not the query text includes a medical issue through the medical assistance model;
wherein, when the query text includes the medical issue, the processing device, through the medical assistance model and based on the medical-related dataset and the health data, determines one or more of at least one related disease, at least one related symptom and at least one related medical department that are associated with the medical issue, and generates a response text.

2. The medical consultation system according to claim 1, wherein the health data includes a plurality of pieces of biometric data, the processing device is further configured to establish a plurality of personalized normal ranges based on the plurality of pieces of biometric data, and the processing device is further configured to, based on the plurality of personalized normal ranges, adjust a determination criterion for determining the one or more of the at least one related disease, the at least one related symptom and the at least one related medical department and generating the response text.

3. The medical consultation system according to claim 2, wherein the processing device is further configured to analyze the health data based on the plurality of personalized normal ranges for determining whether or not an abnormal event occurs;
wherein, when the processing device determines that the abnormal event occurs, an abnormal warning reminder is generated by the at least one user device.

4. The medical consultation system according to claim 2, wherein, when the processing device is further configured to establish the plurality of personalized normal ranges based on the plurality of pieces of biometric data, the processing device is further configured to gather statistics on each one of the plurality of pieces of biometric data within a reasonable variation range to generate one of the plurality of personalized normal ranges that corresponds to the one of the plurality of pieces of biometric data.

5. The medical consultation system according to claim 2, wherein the processing device is further configured to, through a common retrieval-augmented generation model, retrieve medical data associated with the one or more of the at least one related disease, the at least one related symptom and the at least one related medical department from the data warehouse module;
wherein the processing device is further configured to obtain the health data from the personal data module through a personal retrieval-augmented generation model;
wherein the processing device is further configured to, through the medical assistance model, integrate the medical data and the health data for determining the one or more of the at least one related disease, the at least one related symptom and the at least one related medical department and generating the response text.

6. The medical consultation system according to claim 5, wherein the processing device is further configured to, through the common retrieval-augmented generation model, convert the query text into a first semantic vector set, and perform similarity comparison in the medical-related dataset of the data warehouse module based on the first semantic vector set for retrieving a plurality of first data segments related to the medical issue and providing the plurality of first data segments to the medical assistance model.

7. The medical consultation system according to claim 5, wherein the processing device is further configured to, through the personal retrieval-augmented generation model, convert the query text into a second semantic vector set, and perform similarity comparison in the personal data module based on the second semantic vector set for retrieving a plurality of second data segments related to the medical issue and providing the plurality of second data segments to the medical assistance model.

8. The medical consultation system according to claim 6, wherein the processing device is further configured to collect and analyze a plurality of pieces of medical record information to generate a disease-symptom matching dataset, a disease description dataset and a department-disease matching dataset for establishing the medical-related dataset.

9. The medical consultation system according to claim 7, wherein the processing device is further configured to collect a plurality of historical query texts and a plurality of historical response texts for generating and storing a historical conversation dataset in the personal data module, and adjust the determination criterion of the medical assistance model based on the plurality of personalized normal ranges and the historical conversation dataset.

10. The medical consultation system according to claim 9, wherein the processing device is further configured to collect health checkup information and medical history data of the user for establishing and storing a personal health record set in the personal data module, and adjust the determination criterion of the medical assistance model based on the plurality of personalized normal ranges, the historical conversation dataset and the personal health record set.

11. A medical consultation method, comprising processes of:
configuring at least one user device to obtain health data of a user;
configuring a storage device to store a data warehouse module, a personal data module and a medical assistance model, storing a medical-related dataset in the data warehouse module, and storing the health data in the personal data module; and
configuring a processing device electrically connected to the storage device to:
receive a query text inputted by the user;
determine whether or not the query text includes a medical issue through the medical assistance model; and
in response to determining that the query text includes the medical issue, through the medical assistance model and based on the medical-related dataset and the health data, determining one or more of at least one related disease, at least one related symptom and at least one related medical department, and generating a response text.

12. The medical consultation method according to claim 11, wherein the health data includes a plurality of pieces of biometric data, and the medical consultation method further comprising processes of:
establishing a plurality of personalized normal ranges based on the plurality of pieces of biometric data by the processing device; and
adjusting, by the processing device, based on the plurality of personalized normal ranges, a determination criterion for determining the one or more of the at least one related disease, the at least one related symptom and the at least one related medical department and generating the response text.

13. The medical consultation method according to claim 12, further comprising processes of:
analyzing the health data based on the plurality of personalized normal ranges for determining whether or not an abnormal event occurs by the processing device; and
in response to determining that the abnormal event occurs, generating an abnormal warning reminder by the at least one user device.

14. The medical consultation method according to claim 12, wherein the process of establishing the plurality of personalized normal ranges based on the plurality of pieces of biometric data by the processing device includes:
gathering statistics on each one of the plurality of pieces of biometric data within a reasonable variation range to generate one of the plurality of personalized normal range that corresponds to the one of the plurality of pieces of biometric data.

15. The medical consultation method according to claim 12, further comprising processes of:
retrieving, by the processing device, medical data associated with the one or more of the at least one related disease, the at least one related symptom and the at least one related medical department from the data warehouse module through a common retrieval-augmented generation model;
obtaining the health data from the personal data module through a personal retrieval-augmented generation model by the processing device; and
integrating, by the processing device, through the medical assistance model, the medical data and the health data for determining the one or more of the at least one related disease, the at least one related symptom and the at least one related medical department and generating the response text.

16. The medical consultation method according to claim 15, further comprising processes of:
converting the query text into a first semantic vector set through the common search generation model by the processing device; and
performing, by the processing device, through the common search generation model, similarity comparison in the medical-related dataset of the data warehouse module based on the first semantic vector set for retrieving a plurality of first data segments related to the medical issue and providing the plurality of first data segments to the medical assistance model.

17. The medical consultation method according to claim 15, further comprising processes of:
converting the query text into a second semantic vector set through the personal retrieval-augmented generation model by the processing device; and
performing, by the processing device, through the personal retrieval-augmented generation model, similarity comparison in the personal data module based on the second semantic vector set for retrieving a plurality of second data segments related to the medical issue and providing the plurality of second data segments to the medical assistance model.

18. The medical consultation method according to claim 16, further comprising a process of:
collecting and analyzing, by the processing device, a plurality of pieces of medical record information to generate a disease-symptom matching dataset, a disease description dataset and a department-disease matching dataset for establishing the medical-related dataset.

19. The medical consultation method according to claim 17, further comprising processes of:
collecting, by the processing device, a plurality of historical query texts and a plurality of historical response texts for generating and storing a historical conversation dataset in the personal data module; and
adjusting, by the processing device, the determination criterion of the medical assistance model based on the plurality of personalized normal ranges and the historical conversation dataset.

20. The medical consultation method according to claim 19, further comprising processes of:
collecting, by the processing device, health checkup information and medical history data of the user for establishing and storing a personal health record set in the personal data module; and
adjusting, by the processing device, the determination criterion of the medical assistance model based on the plurality of personalized normal ranges, the historical conversation dataset and the personal health record set.
